(19) United States
(12) Patent Application Publication
(10) Pub. No.: US 2023/0302452 A1
(43) Pub. Date: Sep. 28, 2023

(54) IMPROVEMENTS IN OR RELATING TO A METHOD OF MAINTAINING A MICRODROPLET

(71) Applicant: LIGHTCAST DISCOVERY LTD, Cambridge, Cambridgeshire (GB)

(72) Inventors: Nele DIECKMANN, Cambridge, Cambridgeshire (GB); Gianmarco MASTROGIOVANNI, Cambridge, Cambridgeshire (GB); Emma Louise TALBOT, Cambridge, Cambridgeshire (GB); William Michael DEACON, Cambridge, Cambridgeshire (GB)

(73) Assignee: LIGHTCAST DISCOVERY LTD, Cambridge, Cambridgeshire (GB)

(21) Appl. No.: 18/020,503

(22) PCT Filed: Aug. 10, 2021
(86) PCT No.: PCT/GB2021/052068

§ 371 Date: Feb. 9, 2023

(30) Foreign Application Priority Data
Aug. 11, 2020 (GB) 2012485.5

(51) Int. Cl. B01L 3/00; C12M 1/12

(52) U.S. Cl. CPC B01L 3/502784; C12M 25/01; B01L 2200/0673

(57) ABSTRACT

A method of maintaining at least one component in an aqueous microdroplet dispersed in conditioned oil to form an emulsion is provided. The method comprising the steps of supplementing an unconditioned oil with at least one component to form a conditioned oil; and providing the aqueous microdroplet comprising at least one component, wherein the microdroplet is dispersed in the conditioned oil to form an emulsion, such that the partitioning of the component from the microdroplet into the conditioned oil is reduced, wherein the maintenance of the component within the microdroplet is based on the partition coefficient value of the component being equal to or more than zero; or equilibrating the unconditioned oil with a media or a buffer containing at least one component to form the conditioned oil, such that the partitioning of the component from the aqueous microdroplet into the conditioned oil is reduced, wherein the maintenance of the component within the microdroplet is based on the concentration of the component in the conditioned oil being equivalent to or in excess of the product of the partition coefficient and the concentration of the component in the microdroplet. A method of method of making conditioned oil is also provided.

### FIELD OF THE INVENTION

This invention relates to a method of maintaining at least one component in a microdroplet and in particular, a method of maintaining at least one component in an aqueous microdroplet dispersed in conditioned oil to form an emulsion. The invention also relates to a method of making conditioned oil.

### BACKGROUND

There have been known methods in which biological components such as cells, enzymes, oligonucleotides and even single nucleotides are manipulated within microdroplets for the purposes of carrying out a range of analyses including DNA and RNA sequencing and the detection and characterisation of cells and viruses. These methods involve translocating microdroplets dispersed in an immiscible carrier fluid along microfluidic pathways in an analytical device using electrowetting propulsive forces or by directly printing of the microdroplets onto a substrate coated with the carrier fluid.

This requires an emulsion of media components in carrier oil, optionally containing biological components, to be stored on the device. In an emulsion there is often no media exchange and cells are limited to the media components inside their droplet. Similarly, toxic waste products build up inside the droplets over time, limiting cell lifetime. Commonly known instruments where droplets are incubated or stored in channels often have poor levels of cell viability owing to restricted supply of gases. Gas can be exchanged via equilibration of oil and flow of that oil through the device.

Some media may comprise one or more components such as vitamins, salts, amino acids, nutrient buffer components e.g. pH buffer and FBS (fetal bovine serum) components such as growth factors, are vital for cell proliferation and metabolism. These key components such as vitamins allow for in-vitro cell culture but there is a preference for some of these key components to partition heavily into the oil. Such components are then unavailable to the cells within the droplets.

Each of the media components has an associated, well-documented oil:water such as octanol:water partition coefficient which describes the relative concentration of that component in the oil relative to the water at equilibrium.

Any component with a non-zero partition coefficient will have a distribution of that component between the oil and media phase, and therefore a fraction of the component will partition into the oil. Hence, there will be a loss of components within the microdroplets.

Any components with a partition coefficient in excess of 1 will heavily partition into the oil, and so losses of these components will be large. In addition, where the microdroplets are incubated or stored in channels, the cell viability timescale is also lowered owing to a restricted supply of gases. Gases can be exchanged via equilibrium of oil and flow of that oil through the device.

More recently, a device has been developed in which microdroplets can be held in place in an oil flow by optically-mediated electrowetting-on-device (oEWOD). This can still result in some fraction of each media component in the droplets to move into the oil. The amount of components that move from the microdroplet and into the oil can be determined by the partition coefficient of the component. Components partitioned in oil will be carried away from the microdroplets with the oil flow, and subsequently lost. Hence, an oil flow intended to replenish gas, and improve cell viability timescale, can reduce cell viability by resulting in the loss of media from the microdroplets.

Since the partition coefficient is a concentration ratio, a larger molar fraction of the component will be lost from the droplet if the relative oil volume is larger than the droplet volume. As such simply carrying out viability assays in an emulsion with varied oil:water volume or equivalently an array with different droplet spacing can give a wide variation in cell viability. Similar limitations occur for alternative assays that use small molecules e.g. drugs.

Therefore, without addressing the partitioning issue of the components initially contained within aqueous microdroplets, many key components can be lost simply during loading of droplets onto a device. Thus, this can have a detrimental impact on the viability of cells contained within the microdroplets.

It is against this background that the present invention has arisen.

### SUMMARY OF INVENTION

According to an aspect of the present invention, there is provided a method of maintaining at least one component in an aqueous microdroplet, the method comprising the steps of:
supplementing an unconditioned oil with at least one component to form a conditioned oil; and
providing the aqueous microdroplet comprising at least one component, wherein the microdroplet is dispersed in the conditioned oil to form an emulsion, such that the partitioning of the component from the microdroplet into the conditioned oil is reduced,
wherein the maintenance of the component within the microdroplet is based on the partition coefficient value of the component being equal to or more than zero; or
equilibrating the unconditioned oil with a media or a buffer containing at least one component to form the conditioned oil, such that the partitioning of the component from the aqueous microdroplet into the conditioned oil is reduced,
wherein the maintenance of the component within the microdroplet is based on the concentration of the component in the conditioned oil being equivalent to or in excess of the product of the partition coefficient and the concentration of the component in the microdroplet.

As disclosed in the present invention, and unless otherwise stated, the term “product” refers to the multiplication of the named coefficients and concentrations.

According to another aspect of the present invention, there is provided a method of maintaining at least one component in an aqueous microdroplet dispersed in conditioned oil to form an emulsion, the method comprising the step of
supplementing an unconditioned oil with at least one component having a partition coefficient value that is equal to or more than zero to form the conditioned oil, or
equilibrating the unconditioned oil with a media containing at least one component, such that the partitioning of at least one component from the media into the unconditioned oil forms the conditioned oil, wherein the concentration of at least one component in the conditioned oil is equivalent to or in excess to the product of the partition coefficient and the concentration of at least one component in the microdroplet; and
forming the emulsion comprising the conditioned oil and the aqueous microdroplet.

Using conditioned oil to form the emulsion prevents partitioning of the media components into the oil phase, and they therefore remain accessible within the droplets. Hence, this reduces the component from partitioning out of the microdroplets and into the conditioned oil.

Thus, the methods provided herein can be advantageous as it can be used to maintain concentration of at least one component within the microdroplet which may vital for example for cell productivity e.g. improving protein expression and/or secretion rates, cell viability and proliferation within the microdroplet. If a droplet containing media components was put into unconditioned oil, component loss from the droplet would occur.

The media may comprise one or more cells, chemical reagents and/or non-media components. Additionally or alternatively, the media may comprise, but is not limited to, one or more cells, chemical reagents and/or one or more non-media components such as enzymes, fluorescent dyes, reporter agents e.g. primary antibodies and fluorescent dye-conjugated detection antibodies, beads, polymers e.g. PEG, polymers or oligonucleotides.

Some media may include, but is not limited to, one or more of the following: vitamins, salts, amino acids, nutrient buffer components e.g. pH buffer and FBS (fetal bovine serum) components such as growth factors, are vital for cell proliferation and metabolism. Therefore it is important to maintain media concentrations in droplets to prolong the viability of any cells contained within.

The components provided herein may be, but is not limited to vitamins, salts, amino acids and/or nutrients. These components are often required for cell proliferation and metabolism.

The concentration of the components provided in the media can be dependent on the partitioning coefficient of the component. For example, a component with a high partitioning coefficient value may move into the oil readily and therefore, the concentration of the component in the media may need >1× to balance out the partitioning effect of the component. In essence, the components are balanced such that after partitioning of said component the microdroplet retains equivalent of 1× media concentration.

Any component with a non-zero partition coefficient will have a distribution of the component between the oil and media phase i.e. some losses if a droplet was put in unconditioned oil. Those with a partition coefficient in excess of 1 will prefer the oil and so losses will be much heavier.

In some embodiments, the media components has an associated partition coefficient value in oil:water such as octanol:water. In octanol:water, examples of media components which can be lost through partitioning include vitamins such as Biotin (P=2.45), para aminobenzoic acid (P=6.76) and niacinamide (P=0.417), which are essential vitamins for in-vitro culture. Thus, the method of providing the conditioned oil as disclosed in the present invention can significantly help reduce key components partitioning out of the microdroplet and into the oil.

Furthermore, magnesium sulfate (P=0.123) is an enzyme cofactor and a counter ion for ATP and nucleic acids that is important for cell growth. Essential amino acids are required to prevent cell apoptosis including L-Isoleucine, L-Leucine, L-Methionine, L-Phenylanaline, L-Tryptophan, which all have partition coefficients above 0.01, and therefore a fraction of these components can be lost through partitioning if a droplet containing these media components was placed into unconditioned oil.

The partition coefficient (P) can be defined as P=Co/Cw. The Partition coefficient (P)=concentration in oil phase (Co)/concentration in aqueous phase (Cw).

In some embodiments, the at least one component in the media to unconditioned oil ratio is 1:1 during formulation of the conditioned oil. In some embodiments, the at least one component in the media to unconditioned oil ratio is 2:1 or above during formulation of the conditioned oil. This enables effective balancing of the media components in the conditioned oil compared to the media components in the droplets, and prevents partitioning of the media components into the oil phase when droplets and conditioned oil are combined.

In some embodiments, the microdroplet may comprise one or more biological cells. In some embodiments, the microdroplet may be free of biological cells.

In some embodiments, the method may further comprise the microdroplet and/or the conditioned oil containing at least one reagent. An example of a reagent can be biologics. Typically, the reagent can be provided to help proliferation or increase metabolism of cells within the microdroplet.

In some embodiments, the conditioned oil can be equilibrated with O₂ and CO₂. Usually, a concentration of 5% CO₂ (rest air) can be provided for microdroplets containing cells. In other embodiments, hybridomas may use 7% CO₂. This is to match the media and maintain pH. Typical range is around pH 6-8.

Providing O₂ and CO₂ is advantageous because CO₂ can be used to help pH regulation of the conditioned oil and O₂ is required for cell respiration.

In some embodiments, the media can be cell growth media and is selected from one or more of the following; RPMI 1640, EMEM, DMEM, Ham's F12, Ham's F10, F12-K, HAT Medium, or modified versions thereof.

In some embodiments, the media may include additional growth factors or supplements intended to aid cell viability, proliferation and/or productivity.

In some embodiments, one or more microdroplets may contain chemical reagents. In some embodiments, one or more microdroplets may contain beads or reporter agents. In some embodiments, one or more microdroplets may contain stains for facilitating continued staining of cells.

In some embodiments, the method of the present invention may, further comprise the step of loading one or more microdroplets dispersed in the conditioned oil into a EWOD or oEWOD device.

In some embodiments, the microdroplets may be formed within a microfluidic device such as a EWOD or oEWOD device i.e. by flowing bulk media into conditioned oil for example, flow focusing or step emulsification on chip.

In some embodiments, the oEWOD device comprises: a first and a second composite wall. Each of the first and second composite walls comprises a substrate on which a conductor layer is provided. The first composite wall has a photoactive layer on the conductor layer. Each of the first and second composite walls has a continuous dielectric layer that has a thickness of less than 20 nm. The first dielectric layer is provided on the photoactive layer of the first composite wall. The second dielectric layer is provided on the conductor layer of the second composite wall.

The first substrate and the first conductor layer and/or the second substrate and the second conductor layer may be transparent.

The device may further comprise an NC source to provide a voltage across the first and second composite walls connecting the first and second conductor layers; at least one source of electromagnetic radiation having an energy higher than the bandgap of the photoexcitable layer adapted to impinge on the photoactive layer to induce corresponding ephemeral electrowetting locations on the surface of the first dielectric layer; and a microprocessor for manipulating the points of impingement of the electromagnetic radiation on the photoactive layer so as to vary the disposition of the ephemeral electrowetting locations thereby creating at least one electrowetting pathway along which the microdroplet may be caused to move.

The device may further comprise an interstitial layer of silicon oxide. The advantage of the interstitial layer is that it can be used as a binding layer for a anti or non-fouling layer. The interstitial layer is provided between the dielectric layer and the hydrophobic layer. The thickness of the interstitial layer may be between 0.1 nm to 5 nm. The thickness of the interstitial layer can be more than 0.1, 0.25, 0.5, 0.75, 1, 1.5, 2, 2.5, 3, 3.5, 4 or 4.5 nm, or it may be less than 5 nm, 4.5, 4, 3.5, 3, 2.5, 2, 1.5, 1, 0.75, 0.5 or 0.25 nm.

The exposed surfaces of the first and second dielectric layers may be disposed less than 200 μm apart to define a microfluidic space adapted to contain the microdroplet. The microfluidic space may be between 2 and 50 μm in width. In some embodiments, the microfluidic space is more than 2, 4, 6, 8, 10, 12, 14, 16, 18, 20, 22, 24, 26, 28, 30, 32, 34, 36, 38, 40, 42, 44, 46 or 48 μm. In some embodiments, the microfluidic space may be less than 50, 48, 46, 44, 42, 40, 38, 36, 34, 32, 30, 28, 26, 24, 22, 20, 18, 16, 14, 12, 10, 8, 6 or 4 μm.

The exposed surfaces of the first and second dielectric layers may include one or more spacers for holding the first and second walls apart by a predetermined amount to define a microfluidic space adapted to contain the microdroplet. The physical shape of the spacers may be used to aid the splitting, merging and elongation of microdroplets in the device.

In some embodiments, the microfluidic chip of the present invention comprises oEWOD structures comprised of:
a first composite wall comprised of:
   a first substrate
   a first transparent conductor layer on the substrate, the first transparent conductor layer having a thickness in the range 70 to 250 nm;
   a photoactive layer activated by electromagnetic radiation in the wavelength range 400-1000 nm on the conductor layer, the photoactive layer having a thickness in the range 300-1500 nm and
   a first dielectric layer on the photoactive layer, the first dielectric layer having a thickness in the range 30 to 160 nm;
a second composite wall comprised of:
   a second substrate;
   a second conductor layer on the substrate, the second conductor layer having a thickness in the range 70 to 250 nm and
   optionally a second dielectric layer on the second conductor layer, the second dielectric layer having a thickness in the range 30 to 160 nm or 120 to 160 nm
wherein the exposed surfaces of the first and second dielectric layers are disposed less than 180 μm apart to define a microfluidic space adapted to contain microdroplets;
an NC source to provide a voltage across the first and second composite walls connecting the first and second conductor layers;
at least one source of electromagnetic radiation having an energy higher than the bandgap of the photoactive layer adapted to impinge on the photoactive layer to induce corresponding virtual electrowetting locations on the surface of the first dielectric layer; and
means for manipulating the points of impingement of the electromagnetic radiation on the photoactive layer so as to vary the disposition of the virtual electrowetting locations thereby creating at least one electrowetting pathway along which the microdroplets may be caused to move.

In some embodiments, the first and the second dielectric layers may be composed of a single dielectric material or it may be a composite of two or more dielectric materials. The dielectric layers may be made from, but is not limited to, Al₂O₃ and SiO₂.

In some embodiments, a structure may be provided between the first and second dielectric layers. The structure between the first and second dielectric layers can be made of, but is not limited to, epoxy, polymer, silicon or glass, or mixtures or composites thereof, with straight, angled, curved or micro-structured walls/faces.

The structure between the first and second dielectric layers may be connected to the top and bottom composite walls to create a sealed microfluidic device and define the channels and regions within the device. The structure may occupy the gap between the two composite walls.

In some embodiments, the method further comprises the step of introducing a replacement carrier fluid into the device, wherein the replacement carrier fluid is conditioned oil.

The step of introducing a replacement carrier fluid into the device prevents the build-up of any toxic waste products excreted by any biological components within the droplets as a result of metabolic pathways. If toxic waste products build up inside the droplets over time, cell lifetime is limited. Waste components such as lactate (P=0.19) and ammonia (P=1.70) in octanol:water for example, can partition into oil, and can therefore be removed by flow of oil.

In some embodiments, the microdroplet may comprise a release agent. The release agent may be one or more of the following; trypsin, EDTA, protease, citric acid or Accutase.

In some embodiments, method further comprises the step of incubating the microdroplets.

In some embodiments, the method may further comprise the step of monitoring the microdroplet for cell growth.

In some embodiments, the method may further comprise the step of performing a cell assay such as screening cells.

In some embodiments, the method may further comprise one or more of the following steps: merging the microdroplets, splitting the microdroplets and/or dispensing the microdroplets.

In some embodiments, the conditioned oil may be selected from a mineral oil, a silicone oil or a fluorocarbon oil.

In some embodiments, the component is a biological component, a small molecule or a compound.

In some embodiments, the unconditioned oil may contain a surfactant.

According to another aspect of the invention, there is provided a method of making conditioned oil, the method comprising the step of
mixing an unconditioned oil together with aqueous media containing at least one component to form an emulsion comprising the conditioned oil at a desired temperature for use of the conditioned oil in a droplet formation, wherein the mixing enables the partitioning of at least one component to occur into the unconditioned oil to form the conditioned oil; and
recovering and/or separating the conditioned oil following partitioning of the component, wherein the concentration of the media is determined by the desired concentration based on the coefficient value of the component to maintain the partitioning co-efficient value of the component in a subsequent droplet formation.

According to a further aspect of the present invention, there is provided a method of droplet formation comprising a conditioned oil according to any one of the preceding claims, wherein the conditioned oil mixed together with aqueous media forms an emulsion at a desired temperature of use of conditioned oil in droplet formation, recovering/separating the conditioned oil following partitioning, wherein the concentration of the media is determined by the desired concentration to maintain the partition co-efficient of at least one component during and after droplet formation.

The unconditioned oil may contain a surfactant.

According to an aspect of the present invention, there is provided a conditioned oil obtainable by the process according any aspects of the present invention.

According to another aspect of the present invention, there is provided a kit of parts comprising the conditioned oil as prepared and formulated according to any of the previous aspects of the present invention. The kit of parts may further comprise media, which can be cell media. The kit of parts may further comprise an oil or an oil/surfactant mixture. The kit of parts may further comprise one or more components according to any of the previous aspects of the present invention.

In some embodiments, the kit may also comprise one or more non-media components. For example, non-media components may include, but is not limited to, one or more of the following: enzymes, fluorescent dyes, reporter agents e.g. primary antibodies and fluorescent dye-conjugated detection antibodies, beads, polymers e.g. PEG, polymers, oligonucleotides and others.

The kit may also comprise ingredients for a user to condition the oil prior to use.

In some embodiments, the kit may comprise a control such as purified antibodies.

In some embodiments, the conditioned oil may contain at least one reagent. An example of a reagent can be biologics. Typically, the reagent can be provided to help proliferation or increase metabolism of cells within a microdroplet. Additionally or alternatively, the media may comprise one or more cells, chemical reagents and/or non-media components.

### BRIEF DESCRIPTION OF THE FIGURES

The invention will now be further and more particularly described, by way of example only, and with reference to the accompanying drawings, in which:

FIG. 1 provides a set of examples of theoretical results which illustrates how the amino acid concentration within droplets diminishes over time in the device under oil flow;

FIG. 2 provides a set of theoretical results illustrating a higher partition coefficient of a component results in a greater decrease in concentration of that component with oil flow;

FIG. 3 provides a set of theoretical results that shows a higher oil flow rate results in a faster removal of media components from the device;

FIG. 4 provides a set of experimental results of Jurkat viability over a period of time, showing that overbalancing nutrients can be detrimental to cell health;

FIG. 5 provides a set of experimental results that shows improvement in viability of Jurkat cells in conditioned oil, and when oil volume is not in excess of the emulsion;

FIG. 6 provides a set of experimental results that illustrates reduced cell apoptosis with the use of conditioned oil;

FIG. 7 provides a set of experimental results that illustrates oil flow is detrimental to cell viability when partitioning is not correctly balanced;

FIG. 8 shows an example configuration for carrying out the method of the present invention on a microfluidic chip;

FIG. 9 is a graph showing improvement in cell viability when in conditioned oil;

FIG. 10 provides an example showing an improvement in cell viability; and

FIG. 11 shows fluorescein in conditioned oil.

The present invention herein discloses a method of maintaining at least one component in an aqueous microdroplet dispersed in conditioned oil to form an emulsion. The method may comprise the step of supplementing an unconditioned oil with at least one component having a partition coefficient value that is equal to or more than zero to form the conditioned oil.

Alternatively, the conditioned oil may be prepared by equilibrating the unconditioned oil with a media containing at least one component, such that the partitioning of at least one component from the media into the unconditioned oil forms the conditioned oil. The concentration ratio of at least one component in the conditioned oil is equivalent to or in excess to the product of the partition coefficient and the concentration of at least one component in the microdroplet.

Additionally, the temperature can be controlled during the equilibration process, i.e. equilibrating the unconditioned oil with media containing at least one component, such that the rate of partitioning or transfer of components from the microdroplets into the conditioned oil can, to a certain extent, be controlled. In one example, controlling the temperature during the equilibration process can be advantageous as it can improve the efficiency of cell growth within the microdroplets. As another example, the temperature can be increased and/or decreased during the equilibration process to influence the yield of cell growth within the microdroplet.

In some embodiments, the conditioned oil can be stored long-term following equilibration at room temperature or below −20° C. In some embodiments, the recovery yield of the conditioned oil following the equilibration process as described herein can depend on the amount of emulsion formed during the equilibration process.

In some instances, at least one component in the media to unconditioned oil ratio is 1:1 v/v during conditioned oil formulation. Alternatively, at least one component in the media to unconditioned oil ratio is 2:1 v/v or above during conditioned oil formation. In some instances, the component in the media to unconditioned oil ratio may be 3:1, 4:1, 5:1, 6:1, 7:1; 8:1, 9:1, 10:1, 15:1, 20:1, 25:1, 30:1, 35:1, 40:1, 45:1, 50:1, 60:1, 70:1, 80:1, 90:1 or 100:1. The ratio between the media to unconditioned oil may be determined by a volume ratio or it may be determined by a mass ratio. Subsequently, the ratio of one component in the media to conditioned oil can be 1:1 or 2:1 or it may be 3:1, 4:1, 5:1, 6:1, 7:1; 8:1, 9:1, 10:1, 15:1, 20:1, 25:1, 30:1, 35:1, 40:1, 45:1, 50:1, 60:1, 70:1, 80:1, 90:1 or 100:1 v/v.

The concentration ratio of media to unconditioned oil may be determined using the equation below

Equation to calculate initial media concentration needed:

Balanced media x=[1+V_{c}/V_{w}]P
Where 1× will be the microdroplet media concentration.

P=partition coefficient=C_{c}/C_{w}
Vₒ, V_{w}=volume oil, water phase
Cₒ, C_{w}=concentration of component in oil, water phase

As an example only, a partition coefficient of 1 in 1:1 oil:water mixture needs 2× media for balancing conditioned oil.

The method of preparing conditioned oil may comprise the following steps of mixing an unconditioned oil together with aqueous media containing at least one component to form an emulsion comprising the conditioned oil at a desired temperature for use of the conditioned oil in a droplet formation, wherein the mixing enables the partitioning of at least one component to occur into the unconditioned oil to form the conditioned oil. Mixing can further aid partitioning by increasing interfacial surface area.

In a further step of the present invention, a step of mixing an unconditioned oil such as mineral oil, silicone oil or fluorocarbon oil together with aqueous media or water and media powder containing at least one component can occur, where the unconditioned oil may contain a surfactant.

In some embodiments, the character of the interface between the oil and aqueous phases may help reduce the partitioning of the components from the microdroplets into the oil. For example, the surfactant type and concentration, or protein arrangement at the interface influences transport and transport rate from the aqueous phase into the oil.

Other surface-active components for example BSA, can also influence the transport of one or more components out of the microdroplet's aqueous phase and into the oil phase. BSA is present in e.g. fetal bovine serum in the cell media. As long as the oil is equilibrated with the same media that is to be subsequently used, the balance can be maintained.

In some embodiments, during the droplet generation process, the droplets can be placed in a high concentration of a surfactant oil followed by transfer into another lower concentration surfactant equilibrated oil. Exchange of various components can happen so long as the oil has been equilibrated with the correct surfactant concentration. Mixing two conditioned oils of different surfactant concentrations can help maintain balance of components exchanging between the microdroplets and the oil.

The transfer of microdroplets into a lower surfactant concentration can increase retention within the microdroplets. This means that fewer components within the microdroplets partition out.

As referred to herein and unless otherwise specified, a low surfactant concentration is referred to as a concentration below or close to the critical micelle concentration. A high surfactant concentration means a concentration that is equal to or exceeding twice the critical micelle concentration.

The critical micelle concentration (CMC) can be defined as the concentration of surfactants above which micelles form and all additional surfactants added to the system will form micelles.

A component may be, but is not limited to, vitamins, salts, amino acids, nutrients and buffer components, such as pH buffer, suitable to balance reagents inside droplets. The oil can also be equilibrated with O₂ or CO₂ to balance pH and facilitate cell respiration. In some embodiments, the component may be FBS (fetal bovine serum) component such as growth factors.

Within the context of the present invention as disclosed herein and unless otherwise specified, the term “mixing” refers to fluids such as oil and water being in contact.

This can be achieved by either supplementing the unconditioned oil with key components such as amino acids, proteins, nutrients or gases which have a high partition coefficient. A high partition coefficient is considered to be any component with a partition coefficient equal to or more than zero, which will distribute between the oil and aqueous phase.

In some embodiments, the partition coefficient value of zero would indicate that there is little or no partitioning of components. Hence, there may be no or little loss of components moving out from the microdroplet and into the unconditioned oil. In some embodiments, a partition coefficient value of between 0 to 1 for example, 2, 3, 4, 5, 6, 7, 8 or 9 indicates medium to high partitioning of a component. Hence, there may be some medium activity of movement of components moving out of the microdroplet and into the unconditioned oil. In some embodiments, a partition coefficient value of more than 1 may indicate a high partitioning of components. Hence, there may be a substantial loss of components within the microdroplets, since a substantial amount of components can move out from the microdroplet and into the unconditioned oil.

Alternatively, the conditioned oil can be made by making use of the partition effect itself, by adding media containing components to the unconditioned oil sufficient to balance those in the emulsion droplets or an excess of said components. This technique uses the partitioning effect to transfer components into the oil from the media containing components. Additional components can also be added at this stage if desirable e.g. Fetal Bovine Serum (FBS).

The result of mixing the unconditioned oil with aqueous media or water and media powder is an emulsion comprising the conditioned oil at a desired temperature for use of the conditioned oil in a droplet formation. The mixing enables the partitioning of at least one component to occur into the unconditioned oil to form the conditioned oil. Mixing can refer to combining the unconditioned oil and the aqueous phase, and leaving the components to partition. Alternatively, mixing can refer to forcibly combining the two phases together through agitating to disperse them into each other, and increase the boundary surface area between the two phases, which in turn will speed up the partitioning process.

The conditioned oil can be recovered and/or separated following partitioning of the component, wherein the concentration of the media is determined by the desired concentration based on the coefficient value of the component to maintain the partitioning co-efficient value of the component in a subsequent droplet formation. Depending on the densities of the aqueous and oil phases, an upper phase and a lower phase will form, with the conditioned oil forming either the upper or lower phase depending on the densities of the aqueous fluid and the oil used. The separation of the two phases can take place spontaneously, or the process can be sped up by centrifuging at 1000 g for 10 seconds.

An emulsion can be formed. The emulsion may comprise the conditioned oil and the aqueous microdroplet. The microdroplet may be of any suitable shape but preferably, the microdroplet may be spherical. The microdroplet may comprise one or more cells or it may comprise proteins, nucleic acids, polysaccharides, small biologics, beads, small molecules or compounds.

The present invention as disclosed herein also discloses use of the conditioned oil for droplet formation or a method of droplet formation. An emulsion of droplets is prepared comprising the conditioned oil mixed with water or aqueous media at a desired temperature of use of conditioned oil in droplet formation. The droplets can optionally contain cells, and the media used can be cell growth media and can be one or more of the following: RPMI 1640, EMEM, DMEM, Ham's F12, Ham's F10, F12-K, HAT Medium. The desired temperature for use with droplets containing cells is 37° C. Droplets can contain other biological components, small molecules or compounds. The droplets can also optionally contain reagents, or optionally may contain a release agent. The emulsion droplets can be formed using a microfluidic device such as a flow focusing junction or step emulsifier. Media and any additional components including cells and/or beads can be flowed through an emulsifying apparatus to form droplets. Cells or any other components will be dispersed throughout the droplets.

The emulsion droplets are dispersed in the conditioned oil and loaded into an electrowetting (EWOD) or an opto-electrowetting (oEWOD) device. The conditioning of the oil means no further partitioning of the media in the emulsion droplets with the conditioned oil occurs. Droplets can be incubated and/or monitored for cell growth. Droplets can optionally be sorted by the oEWOD device depending on the desired parameters such as cell content or size. Optionally, droplets can be sorted into an array. Droplets can also be merged together or spilt. Another option enables the droplets and their content to be dispensed from the device.

A replacement carrier fluid such as the conditioned oil can be introduced to the device to replenish gas and media. Additionally, since some waste components partition out of the droplets into the oil, the toxic environment built up around any cells which may be contained within droplets held in the device, can be mitigated.

If the replacement carrier fluid were an unconditioned oil partitioning of components from the microdroplets into the oil phase would occur towards reaching thermodynamic equilibrium. In this case, replacement of the carrier fluid to replenish gas would transport components partitioned into the oil out of the device. Further partitioning of components would then occur into the fresh unconditioned oil exacerbating losses. Replacement of a conditioned carrier fluid negates losses of balanced components but still enables transport of unbalanced waste products out of the device.

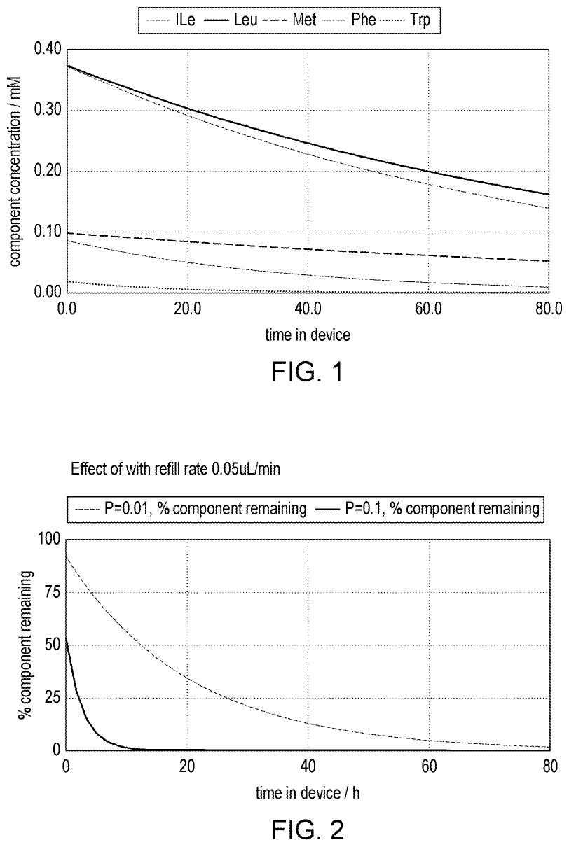

Referring to FIG. 1, there is illustrated an example of components partitioning effect. 1000 droplets containing media are added to a single length device, and the flow of unconditioned oil through the device is controlled at a rate of 0.05 μL/min. The concentration of different amino acids in the droplets on device is modelled over 80 hours. FIG. 1 shows the concentration of all amino acids measured depleting with time in device. FIG. 1 does not account for the use of media by cells and therefore shows the transfer away of amino acids from the microdroplets by oil flow.

Referring to FIG. 2, there is illustrated another example of components partitioning effect. 1000 droplets containing media with partition coefficients of P=0.01 and P=0.1 are added to a single length device, and unconditioned oil is flowed through the device at a rate 0.05 μL/min for 80 hours. The percentage of components remaining in the droplets on device is modelled. FIG. 2 shows that a higher partition coefficient results in a faster depletion of that component from the droplets over time.

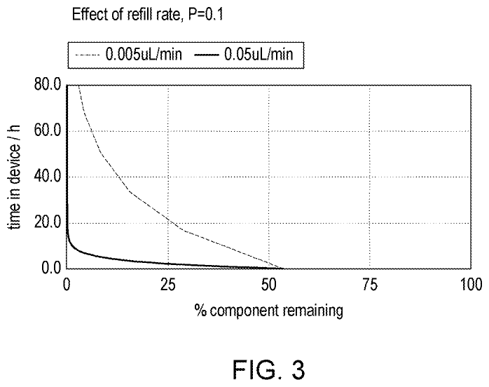

Referring to FIG. 3, there is illustrated a further example of components partitioning effect. 1000 droplets containing media with a partition coefficient of P=0.1 are added to a single length device, and unconditioned oil is flowed through the device at a rate 0.05 μL/min and 0.005 μL/min for 80 hours. The percentage of the measured component remaining in the droplets is modelled. FIG. 3 shows that a higher oil flow rate through the device results in a faster removal of the component from the droplet.

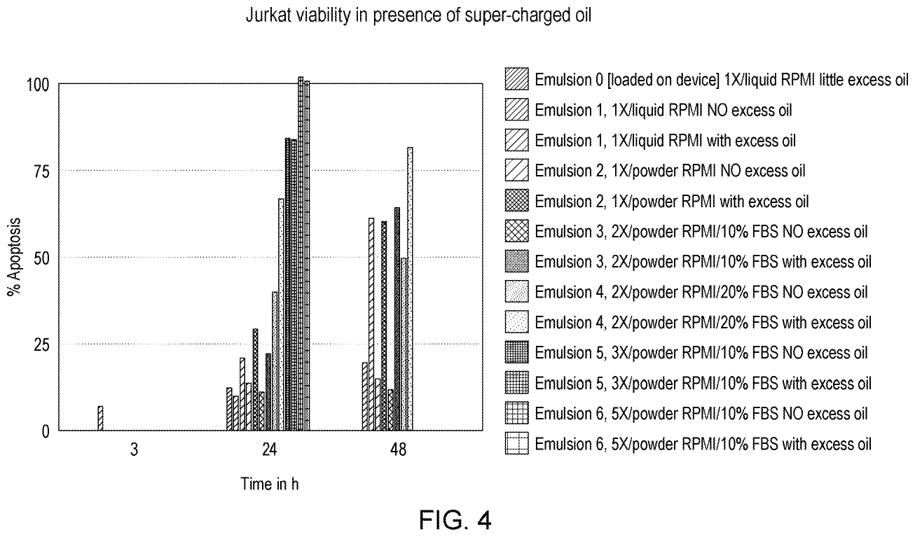

Referring to FIG. 4, there is shown a set of experimental results on Jurkat viability in the presence of conditioned oil over a period of 48 hours. In this experiment, illustrated by FIG. 4, 1×, 2×, 3×, 5× concentration of RPMI+HEPES powder (Thermo Fisher Scientific, UK) is dissolved in sterile water, and where the concentration is relative to the media components in droplets after oil partitioning. As illustrated in FIG. 4, overbalancing of nutrients is shown to be detrimental to cell viability.

The solution is sterile filtered and 10-20 vol. % FBS is added, before filtering again through a 0.2 μm sterile filter. The 6 growth media compositions are as detailed in Table 1.

Table 1 shows the composition of the prepared growth media preparations

| Growth media | RPMI + HEPES | | |
|---|---|---|---|
| preparation | conc. | RPMI type | FBS volume |
| 1 | 1× | liquid | 10% |
| 2 | 1× | powder | 10% |
| 3 | 2× | powder | 10% |
| 4 | 2× | powder | 20% |
| 5 | 3× | powder | 10% |
| 6 | 5× | powder (saturated) | 10% |

The prepared growth media preparations were used to produce conditioned oils by combining two volumes of the prepared growth media with one volume of HFE7500-2% surfactant oil, and rotated overnight. The emulsion was then spun in a centrifuge at 1000 g for 10 s and filtered.

Jurkat WT cells were counted and re-suspended at a concentration of 3M in 1 mL RPMI with added HEPES.

IncuCyte Caspase 3/7 was added at a final concentration of 1 μM (1:5 000 dilution) by first preparing 100 μL of 1:100 dilution in full medium HEPES, and then adding 20 μL of the diluted Caspase to the cells. Hoechst 33342 was added at a final concentration of 250 nM (1:80 000 dilution) by preparing 800 μL of a 1:800 dilution in full medium+HEPES and then by adding 10 μL of the diluted Hoechst to the cells (further 100× dilution). Emulsification followed. All 6 growth media preparations were prepared with a 1.5 mL tube with 50 μL emulsion and 150 μL of oil, and a second 1.5 mL tube with 50 μL of emulsion only. Tubes were sealed and incubated overnight at 37° C.

Analysis was carried out using the automated count followed by manual QC in which at least two field of views (FOV) per condition were checked by eye for quality of count markups. The automated count was found to have an issue with droplet edges fluorescing strongly in the 405/Hoechst channel in some FOVs which can cause false-positive counts in total cell count and thus can artificially reduce the apparent % cell death readout. Where necessary a manual count was performed to refine or even replace automated data.

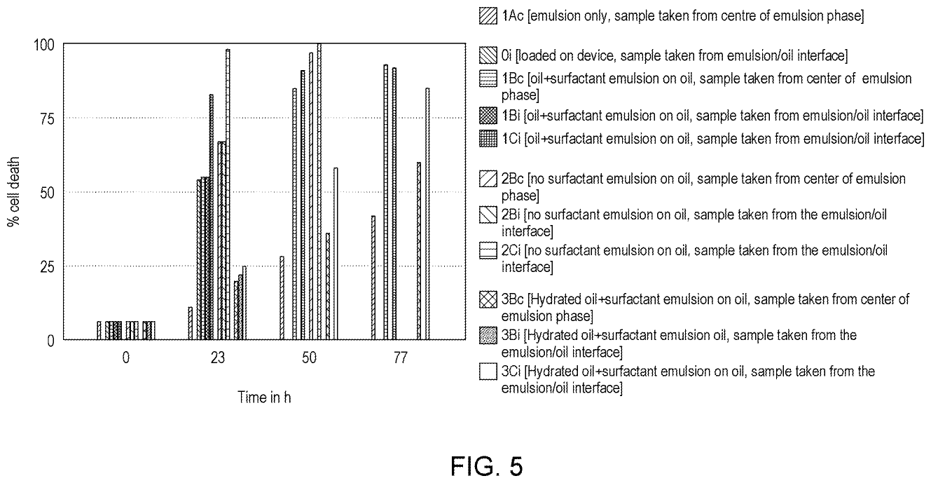

Referring to FIG. 5, there is illustrated an example of how conditioned oil can reduce cell death. The percentage of Jurkat cell death at 0 and 23, 50 and 77 hours. FIG. 5 shows results that represent oil which has been conditioned. Conditioned oil is shown to result in lower percentage cell death compared to unconditioned oil. The viability of Jurkat cells is also determined to be better when oil volume is not in excess of the emulsion, as less components partition out.

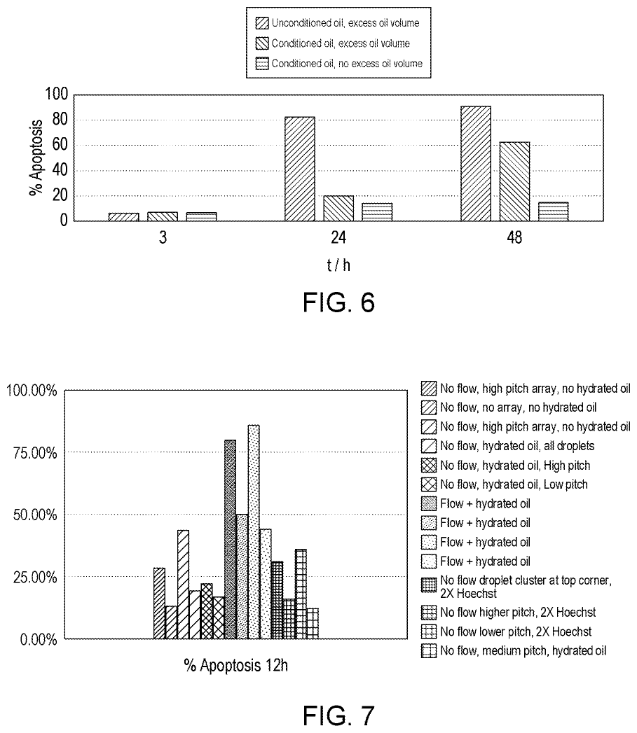

Referring to FIG. 6, there is illustrated another example of components partitioning effect. The percentage of Jurkat cell apoptosis in droplets in an incubator with and without conditioning of the carrier oil is shown. The conditioned oil, with partitioned in components results in a reduced percentage cell apoptosis after 24 and 48 hours compared to unconditioned oil. In addition, the volume of oil being in excess of the volume of emulsion is also varied and shows that the conditioned oil reduces the partitioning effect into this oil reservoir.

Referring to FIG. 7, there is illustrated another example of the effect of incorrectly conditioned/overbalanced oil flow on cell viability. The percentage of Jurkat cell apoptosis after 12 hours on device is shown with and without the flow of conditioned and unconditioned oil. FIG. 7 shows results that represent oil which has been conditioned. When conditioned oil is flowed, there is a higher percentage of cell apoptosis after 12 hours compared to no flow of conditioned oil. This demonstrates that when partitioning is not correctly balanced, oil flow is detrimental to cell viability as it carries components away from droplets and more components then partition out.

The emulsion comprising the conditioned oil and the microdroplet can be loaded into an electrowetting (EWOD) or an opto-electrowetting (oEWOD) device. An example of an electrowetting device can be further described as below.

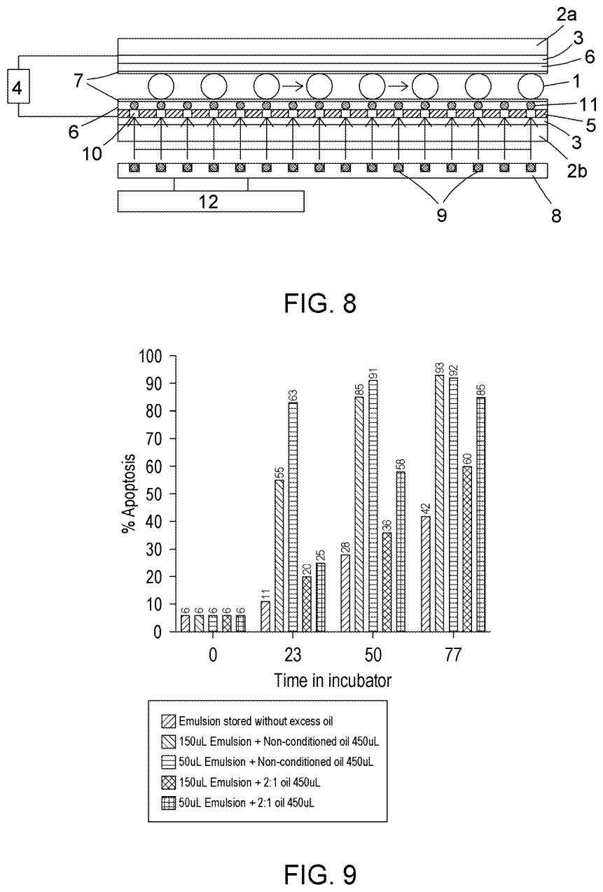

The example oEWOD device as shown in FIG. 8 can be suitable for the manipulation of aqueous microdroplets 1 having been emulsified into a fluorocarbon oil, having a viscosity of 1 centistokes or less at 25° C. and which in their unconfined state have a diameter of less than 200 μm e.g. in the range 20 to 180 μm. In some embodiments, the diameter may be more than 20, 30, 40, 50, 60, 80, 100, 120, 140, 160 or 180 μm. In some embodiments, the diameter may be less than 200, 180, 160, 140, 120, 100, 80, 60, 50, 30, 30 or 20 μm.

The oEWOD stack of the device comprises top 2a and bottom 2b glass plates each 500 μm thick coated with transparent layers of conductive Indium Tin Oxide (ITO) 3 having a thickness of 130 nm. Each of the layers of conductive Indium Tin Oxide (ITO) 3 is connected to an A/C source 4 with the ITO layer on bottom glass plate 2b being the ground. Bottom glass plate 2b is coated with a layer of amorphous silicon 5 which is 800 nm thick. Top glass plate 2a and the layer of amorphous silicon 5 are each coated with a 160 nm thick layer of high purity alumina or Hafnia 6 which are in turn coated with a monolayer of poly(3-(trimethoxysilyl)propyl methacrylate) 7 to render the surfaces of the layer of high purity alumina or Hafnia 6 hydrophobic.

Top glass plate 2a and the layer of amorphous silicon 5 are spaced 8 μm apart using spacers (not shown) so that the microdroplets undergo a degree of compression when introduced into the device cavity. An image of a reflective pixelated screen, illuminated by an LED light source 8 is disposed generally beneath bottom glass plate 2b and visible light (wavelength 660 or 830 nm) at a level of 0.01 Wcm2 is emitted from each light spot 9 and caused to impinge on the layer of amorphous silicon 5 by propagation in the direction of the multiple upward arrows through bottom glass plate 2b and the layer of conductive Indium Tin Oxide (ITO) 3.

At the various points of impingement, photoexcited regions of charge 10 are created in the layer of amorphous silicon 5 which induce modified liquid-solid contact angles on the layer of high purity alumina or Hafnia 6 at corresponding electrowetting locations 11. These modified properties provide the capillary force necessary to propel the microdroplets 1 from one electrowetting location 11 to another. LED light source 8 is controlled by a microprocessor 12 which determines which of the diodes 9 in the array are illuminated at any given time by pre-programmed algorithms.

Referring to FIG. 9, there is shown results of cells in conditioned oil. As shown in FIG. 9, cell death or apoptosis is significantly reduced when the cells have been in conditioned oil compared to cells that are in unconditioned oil. The viability of cells is also determined to be better when oil volume is not in excess of the emulsion, as less components partition out.

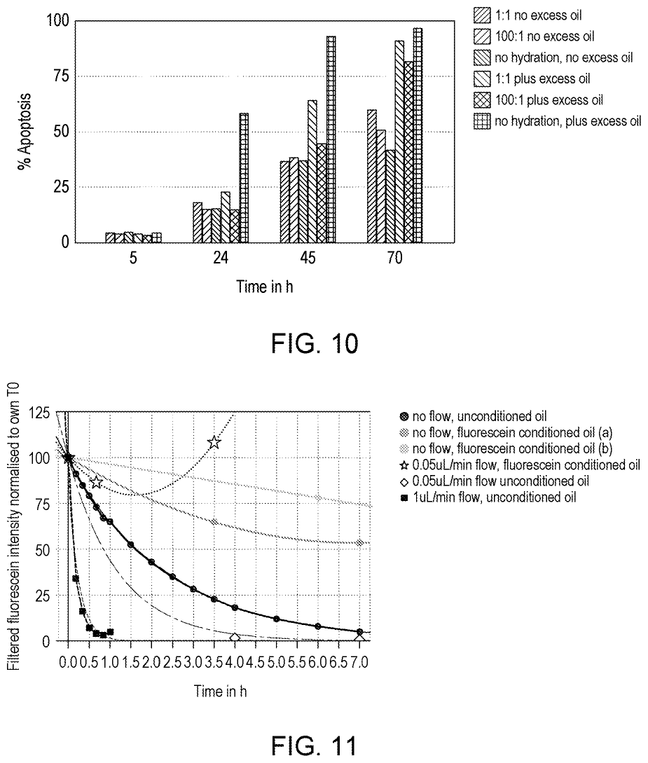

Referring to FIG. 10, there is shown a graph when the droplets containing cells are in oil conditioned (hydrated) with at least one component in the media vs no conditioning/equilibration of components in the media. As shown in FIG. 10, cell death is significantly reduced when the droplets containing cells are in conditioned oil equilibrated with at least one component in the media a ratio of 1:1 v/v. The results also show a further reduction of cell death when the conditioned oil is equilibrated with components in the media at ratio of 100:1 v/v.

Referring to FIG. 11, there is provided a graph showing results of conditioning oil with fluorescein. FIG. 11 shows loaded fluorescein droplet in unconditioned oil and over several hours, the fluorescein leaks out or partitions out of the droplet. In some instances, the flow rate can make a difference to the leakage. For example, faster flow rates can result in an increase in loss of the fluorescein out of the droplet and into the oil. As illustrated in FIG. 11, one or more components and/or fluorescein can be added into the unconditioned oil to form a conditioned oil. When the conditioned oil contains fluorescein, the graph as illustrated in FIG. 11 demonstrates that there is less leakage of the fluorescein from droplets into the conditioned oil. Moreover, droplets can be regularly resupplied with fluorescein and/or other essential components to further reduce the partitioning of fluorescein out of the droplets over time.

As disclosed in any aspect of the invention and/or in any of the embodiments herein, the partitioning of components out of droplets can effectively be controlled when droplets are placed in the conditioned oil as described in the present invention. As an example, the partitioning of components from the microdroplets can be reduced. This invention as described herein can be applicable to many biological and/or chemical workflows such as cell assays and/or chemical reaction assays, where no cells are involved.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

1. A method of maintaining at least one component in an aqueous microdroplet, the method comprising the steps of
supplementing an unconditioned oil with at least one component to form a conditioned oil; and
providing the aqueous microdroplet comprising at least one component, wherein the microdroplet is dispersed in the conditioned oil to form an emulsion, such that the partitioning of the component from the microdroplet into the conditioned oil is reduced,
wherein the maintenance of the component within the microdroplet is based on the partition coefficient value of the component being equal to or more than zero; or
equilibrating the unconditioned oil with a media or a buffer containing at least one component to form the conditioned oil, such that the partitioning of the component from the aqueous microdroplet into the conditioned oil is reduced,
wherein the maintenance of the component within the microdroplet is based on the concentration of the component in the conditioned oil being equivalent to or in excess of the product of the partition coefficient and the concentration of the component in the microdroplet.

2. The method according to claim 1, wherein the at least one component in the media to unconditioned oil ratio is 1:1 v/v when formulating the conditioned oil.

3. The method according to claim 1, wherein the at least one component in the media to unconditioned oil ratio is 2:1 v/v or above when formulating the conditioned oil.

4. (canceled)

5. The method according to claim 1, wherein the conditioned oil is equilibrated with O₂ and CO₂.

6. The method according to claim 1, wherein the microdroplets further comprises at least one biological cell.

7. The method according to claim 1, wherein the media is cell growth media.

8. The method according to claim 7, wherein the cell growth media is selected from one or more of the following; RPMI 1640, EMEM, DMEM, Ham's F12, Ham's F10, F12-K, HAT Medium.

9. The method according to claim 1, further comprising the step of loading one or more microdroplets dispersed in the conditioned oil into a EWOD or oEWOD device.

10. (canceled)

11. The method according to claim 1, further comprises the step of introducing a replacement carrier fluid into the device, wherein the replacement carrier fluid is conditioned oil.

12-13. (canceled)

14. The method according to claim 1, wherein the method further comprises the step of incubating the microdroplets.

15. The method according to claim 1, further comprising the step of monitoring the microdroplet for cell growth.

16. The method according to claim 1, further comprising the step of performing a cell assay.

17. The method according to claim 1, further comprising one or more of the following steps: merging the microdroplets, splitting the microdroplets and/or dispensing the microdroplets.

18. The method according to claim 1, wherein the conditioned oil is selected from a mineral oil, a silicone oil or a fluorocarbon oil.

19. The method according to claim 1, wherein the component is a biological component, a small molecule or a compound.

20. A method of making conditioned oil, the method comprising the step of
mixing an unconditioned oil together with aqueous media containing at least one component to form an emulsion comprising the conditioned oil at a desired temperature for use of the conditioned oil in a droplet formation, wherein the mixing enables the partitioning of at least one component to occur into the unconditioned oil to form the conditioned oil; and
recovering and/or separating the conditioned oil following partitioning of the component, wherein the concentration of the media is determined by the desired concentration based on the coefficient value of the component to maintain the partitioning co-efficient value of the component in a subsequent droplet formation.

21. The method according to claim 20, wherein the at least one component in the media to unconditioned oil ratio is 1:1 v/v.

22. The method according to claim 20, wherein the at least one component in the media to unconditioned oil ratio is 2:1 v/v or above.

23. A method of droplet formation comprising a conditioned oil according to claim 20, wherein the conditioned oil mixed together with aqueous media forms an emulsion at a desired temperature of use of conditioned oil in droplet formation, recovering/separating the conditioned oil following partitioning, wherein the concentration of the media is determined by the desired concentration to maintain the partition co-efficient of at least one component during and after droplet formation.

24. A conditioned oil obtainable by the process according to claim 20.
